(19) United States
(12) Patent Application Publication
(10) Pub. No.: US 2025/0288031 A1
(43) Pub. Date: Sep. 18, 2025

(54) SYSTEMS AND METHODS FOR A SPLIT KNEEPAD CONFIGURED TO REDUCE SCAR-RELATED PAIN

(71) Applicant: Laurel A. Wicks, Jackson, WY (US)

(72) Inventor: Laurel A. Wicks, Jackson, WY (US)

(21) Appl. No.: 18/605,357

(22) Filed: Mar. 14, 2024

(51) Int. Cl. A41D 13/06; A41D 13/015; A61F 5/30

(52) U.S. Cl. CPC A41D 13/065; A41D 13/0153; A61F 5/30

(57) ABSTRACT

A split-kneepad design addresses quality-of-life issues for individuals that have undergone total knee replacement surgery. A pair of cushion inserts with an inner beveled angle that, when worn by the user, are oriented parallel to the axis of the vertical scar resulting from surgery, and are shaped and positioned so that the padding makes contact with the user's knee in regions adjacent to the scar, but do not result in direct compressive force on the scar itself when the user kneels or otherwise applies force to the kneepad.

### TECHNICAL FIELD

The present invention relates, generally, to kneepad technology. More particularly, the present invention relates to a split kneepad design configured to reduce the scar-related pain experienced by individuals after total knee replacement surgery.

### BACKGROUND

Due in part to the aging population, recent years have seen a dramatic increase in knee surgery-particularly total knee replacement surgery. Approximately 600,000 knee replacements were performed in the U.S. in 2019, and that number is expected to grow to 3,000,000 by the year 2030.

Total knee replacement surgery generally involves making a five to eight-inch vertical incision over the front of the knee to expose the joint. Damaged cartilage surfaces at the ends of the femur and tibia are removed along with a specified amount of underlying bone. This is followed by the fitting of metal implants, resurfacing of the patella, and insertion of a plastic spacer.

Post-surgery, the patient is left with a vertical scar over the knee ranging from five to eight inches long. While the swelling, bruising, and redness in the vicinity of the scar reduces to some extent in the weeks after surgery, the scar remains sensitive, particularly to compression of the type that might occur while kneeling, practicing yoga, and the like. The resulting pain can cause significant, long-term quality-of-life issues.

While traditional kneepads may be used to reduce scar-related knee pain, such conventional kneepad designs are unsatisfactory in a number of respects. Most notably, due to the uniform, continuous nature of traditional kneepad padding, the weight of the kneeling individual still results in compressive stress impinging directly on the vertical scar, which is in direct contact with the padding of the knee pad. That is, while traditional kneepads can accommodate a portion of the compressive force through elastic deformation, a significant portion of the stress will always impinge upon the scar itself, resulting in unsatisfactory performance.

There is therefore a long-felt need for kneepad designs that can reduce pain and improve the quality of life for patients after knee-replacement surgery.

### SUMMARY OF THE INVENTION

In accordance with various embodiments, the present invention provides an advanced kneepad design (a Kneezier™ brand kneepad) that can significantly reduce scar-related knee pain after knee-replacement surgery. In particular, a novel, split-kneepad design features a kneepad body enclosing a pair of cushion inserts (e.g., foam core inserts) that, when worn by the user, are oriented parallel to the scar axis, and are shaped and positioned so that—by virtue of a central notched region—make contact with the user's knee in regions adjacent to the scar, but do not result in direct compressive force on the scar itself when the user kneels or otherwise applies force to the kneepad. The kneepad also features a unique “X”-shaped strap configuration that assists in securing the kneepad to the back of the user's knee. Additional features and characteristics will also become apparent from the subsequent detailed description and the appended claims, taken in conjunction with the accompanying drawings.

### BRIEF DESCRIPTION OF THE DRAWING FIGURES

Exemplary embodiments will hereinafter be described in conjunction with the appended drawing figures, wherein like numerals denote like elements, and:

FIGS. 1A-1C are conceptual illustrations showing operation of a split kneepad in accordance with the present invention;

FIGS. 2A-2B are overviews of a kneepad design in accordance with one embodiment of the present invention;

FIGS. 3A-3B depict foam inserts in accordance with one embodiment;

FIG. 4 is a close-up of the padded region of a kneepad in accordance with the embodiment shown in FIG. 2A;

FIG. 5 is an alternative view of the kneepad shown in FIGS. 2A with the strap ends connected; and

FIG. 6 shows exemplary pattern pieces used to fabricate a kneepad in accordance with the present invention.

### DETAILED DESCRIPTION OF PREFERRED EXEMPLARY EMBODIMENTS

The present invention generally relates to a novel split-kneepad design designed to significantly reduce scar-related knee pain after knee-replacement surgery. In that regard, the following detailed description is merely exemplary in nature and is not intended to limit the invention or the application and uses of the invention. Furthermore, there is no intention to be bound by any theory presented in the preceding background or the following detailed description. In the interest of conciseness, commonly known techniques, systems, and methods related to knee surgery, kneepad technology, fabrics, and the like, need not be described herein.

Before providing a detailed description of the invention, it is useful to briefly discuss the geometry of the knee and the general nature of knee-replacement scars. In particular, the two standard incisions used in total knee replacement surgery are the midline incision and the medial parapatellar incision. The midline incision is a substantially straight linear incision made over the top of the patella. The medial parapatellar incision, on the other hand, is curvilinear with lateral concavity extending around the patella region.

The present invention is particularly directed to the scarring produced as a result of a midline incision; however, it will be appreciated that the design principles described herein may also be used to design kneepads that minimize pain for curvilinear scarring, and thus the present invention is not limited to the particular scar geometries described and illustrated in the present application.

With respect to vocabulary, the term “vertical” as used in connection with scar orientation, kneepad components, and the like, will be understood to mean parallel to the midline incision. Furthermore, in some cases, the terms “axial” and/or “scar axis” may be used interchangeably with the term “vertical.” Similarly, the term “lateral” and “laterally” refer to an orientation perpendicular to the vertical axis (e.g., to the left and right of the scar itself). The terms “inside” and “inner” generally refers to a surface facing toward the knee when worn, and the terms “outside” and “outer” generally refer to a surface facing away from the user's knee—i.e., the surface or surfaces likely to be in contact with the ground or other object during use.

In general, as described in further detail below, the present invention relates to a novel split-kneepad design that features a kneepad body enclosing a pair of cushion inserts (e.g., foam core inserts) that, when worn by the user, are oriented parallel to the scar axis, and are shaped and positioned so that they make contact with the user's knee in regions adjacent to the scar, but do not result in direct compressive force on the scar itself when the user kneels or otherwise applies force to the kneepad.

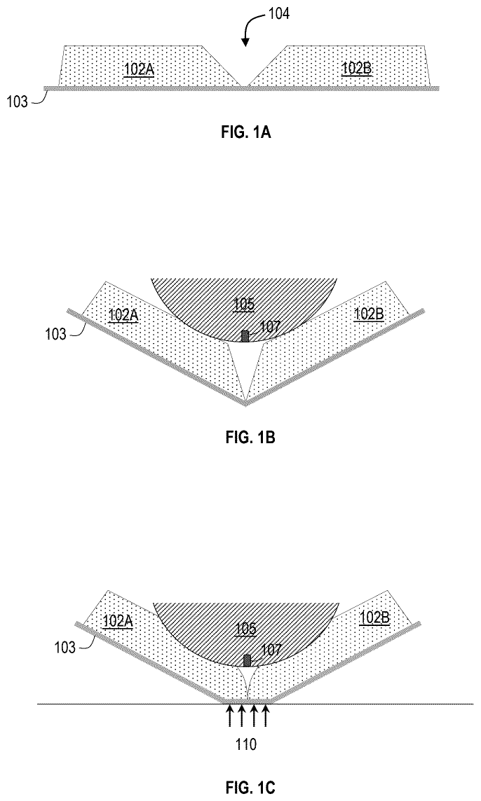

The above functionality is illustrated in the conceptual (not-to-scale) cross-sectional view of FIGS. 1A-1C. That is, FIG. 1A illustrates a portion of the kneepad including the two opposing padded regions 102A and 102B, and a bottom surface 103 of the body, prior to being worn. For simplicity, the individual inserts and body portions are not separately illustrated in this drawing. Note that the inner edges of the padded regions 102A and 102B extend from the base at approximately a 45-degree angle to form a “notched” shape 104. The thickness of the padded regions 102A and 102B, in combination of this inner angle, contribute to the overall effectiveness of the design, as described below.

FIG. 1B illustrates, conceptually, the kneepad being worn, prior to compression, showing the position of the scar region 107 relative to knee 105 and padded regions 102A and 102B (as viewed from above). FIG. 1C shows the same configuration with a force 110 applied to the surface 103 near the center of the kneepad. As can be seen, the padded regions 102A and 102B (each consisting of a cushion insert housed in the kneepad body) deform in response to this force, but not to the extent that they contact the scar region 107.

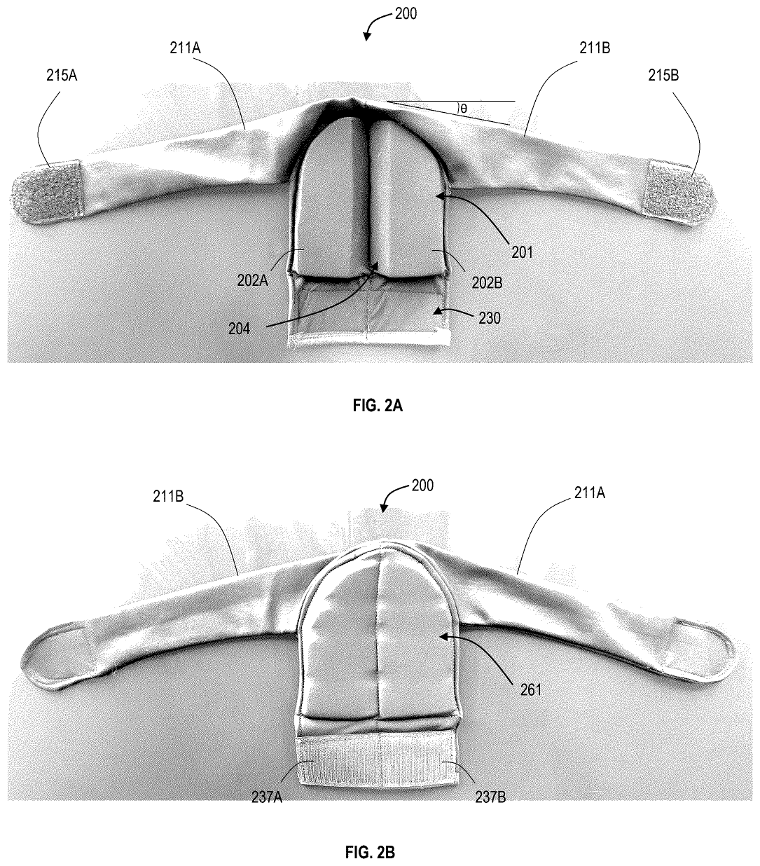

FIGS. 2A and 2B are top views of an example kneepad 200, showing the inner and outer views of the apparatus, respectively. Referring first to FIG. 2A, kneepad 200 includes an inner padded area 201 including padded regions 202A and 202B separated by an inner notch 204 formed by beveled surfaces (e.g., two facing 45-degree angles, combining to form a 90-degree angle when the kneepad lies flat). A rectangular lower fastening region 230 extends downward, and a pair of straps (211A and 211B) extend outward from the top of padded region 201 at a non-zero angle θ of about 10-20 degrees. The straps 211A and 211B include respective hook-and-loop (e.g., Velcro™) regions 215A and 215B at their distal ends.

As shown in FIG. 2B, the lower fastening region 230 includes hook-and-loop surfaces 237A and 237B, which are configured to receive strap ends 215A and 215B, respectively, when straps 211A and 211B are wrapped around the back of the user's knee. It will be appreciated that the angle θ facilitates the crossing of straps 211A and 211B so that it forms an “x” behind the knee.

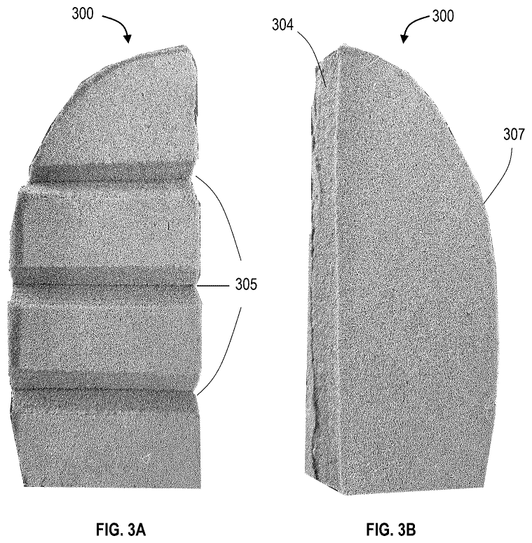

FIGS. 3A and 3B show top and bottom views of an example foam core insert 300 used in connection with padded regions 202A and 202B. That is, FIG. 3A shows the outer surface of insert 300, and FIG. 3B shows the inner surface of insert 300. As illustrated, the foam core insert 300 includes one arcuate side edge 307 opposite a beveled edge 304 (which is angled at about 40-50 degrees-preferably 45 degrees. Also shown are a number of horizontal gouged regions 305 that facilitate bending along the horizontal axis, allowing the kneepad to bend and conform to the shape of the user's knee. The thickness of insert 300 may vary depending upon the structural characteristics and geometry of the foam core insert. In one embodiment, the insert is a one-inch dense foam core material.

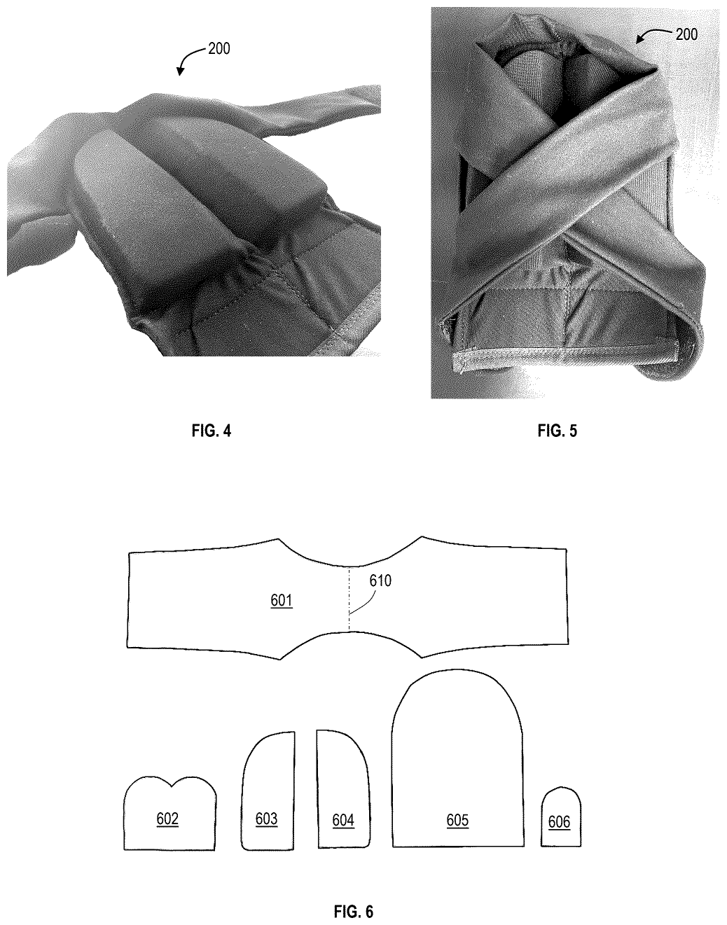

FIG. 4 is an isometric close-up of the padded region of the kneepad 200, and FIG. 5 illustrates a top view of the kneepad 200 with the straps attached (sans knee), illustrating the unique “X”-shaped strap pattern that helps keep the kneepad 200 fastened to the user's knee. While not illustrated in FIG. 5, it will be apparent to one skilled in the art how the user's knee would be positioned within the kneepad 200 assembly shown in the drawings.

It will be appreciated that kneepads in accordance with the present invention may be fabricated using a range of materials and construction techniques known in the art. In that regard, FIG. 6 illustrates a number of pattern pieces that may be used to fabricate the particular embodiment shown in the foregoing drawings. The shapes in FIG. 6 are drawn substantially to scale, but it will be understood that the invention is not limited to the shapes or sizes set forth in FIG. 6.

Pattern piece 601 is a strap piece, which may be cut from one piece of stretch fabric, such as Lycra™. This strap piece is folded along its major (horizontal) axis, and the central vertical axis is used to mark a dart. For scale, the lateral dimension (length) of pattern piece 601 in the illustrated embodiment is approximately 22.5 inches.

Pattern piece 602 is used to fabricate the strap ends (two pieces), which may be fabricated using a suitable fabric, such as Cordura™. The fabric is folded midway along its vertical axis prior to sowing to the strap itself.

Pattern pieces 603 and 604 correspond to the foam core insert, as illustrated above in FIGS. 3A and 3B. That is two pieces of one-inch dense foam are used to form the left and right sides. The interior of the long inner straight sides of each are beveled at approximately a 45-degree angle, and three, quarter-inch-deep horizontal troughs are gouged out to form finished pieces as shown in FIGS. 3A and 3B.

Pattern piece 605 is used to fabricate a two-piece kneepad body, which will encompass the foam core inserts. In one embodiment, one piece comprises Lycra (the inner surface) and one piece comprises Cordura (the outer surface). The lower portion of pattern piece 605 corresponds to region 230 in FIG. 2A. Pattern piece 606 correspond to strap end fastening regions, i.e., regions 215A and 215B in FIG. 2A.

In summary, what has been described is a novel split-kneepad design designed to significantly reduce scar-related knee pain after knee-replacement surgery. It features a central unique “X” shaped strap pattern, and split-kneepad design that, when worn by the user, includes cushioned pads that make contact with the user's knee in regions adjacent to the scar, but do not result in direct compressive force on the scar itself.

In accordance with one embodiment, a kneepad apparatus comprises: a kneepad body having a first side configured to rest against a user's knee, and a second side opposite the first side: a padded area provided on a first side of the kneepad body, the padded area including a first padded region and a second padded region symmetrically situated on opposite sides of a central axis, wherein each of the first and second padded regions include a beveled inner surface adjacent the central axis, such that the first and second padded regions are separated by an inner notch formed by the beveled inner surfaces, and the first and padded regions are hingedly connected at the central axis: a lower fastening region extending downward from a bottom end of the padded area along the central axis: first and second straps extending outward, at a non-zero angle, from a top end of the padded area, wherein the first and second straps include distal strap ends; and a lower fastening region configured to receive the distal strap ends, such that, when wrapped around the back of a user's knee, the inner notch of the padded area is aligned with, but does not contact, a scar on a user's knee.

A method of making a kneepad apparatus in accordance with one embodiment includes: forming, from a single piece of stretch fabric, a first pattern piece; folding the first pattern piece around a pair of foam inserts and sewing the first pattern piece to form a kneepad body having a first side configured to rest against a user's knee, and a second side opposite the first side: wherein the first side of the kneepad body has a padded area including a first padded region and a second padded region symmetrically situated on opposite sides of a central axis, wherein each of the first and second padded regions include a beveled inner surface adjacent the central axis, such that the first and second padded regions are separated by an inner notch formed by the beveled inner surfaces, and the first and padded regions are hingedly connected at the central axis: wherein the body further includes a lower fastening region extending downward from a bottom end of the padded area along the central axis: attaching first and second straps extending outward, at a non-zero angle, from a top end of the padded area, wherein the first and second straps include distal strap ends and are configured to connect to a lower fastening region of the kneepad body such that, when wrapped around the back of a user's knee, the inner notch of the padded area is aligned with, but does not contact, a scar on a user's knee.

While the present invention has been described in the context of the foregoing embodiments, it will be appreciated that the invention is not so limited. As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated. The particular embodiments described above are only examples, and are not intended to limit the scope, applicability, or configuration of the invention in any way. To the contrary, various changes may be made in the function and arrangement of elements described without departing from the scope of the invention.

1. A kneepad apparatus comprising:
a kneepad body having a first side configured to rest against a user's knee, and a second side opposite the first side;
a padded area provided on a first side of the kneepad body, the padded area including a first padded region and a second padded region symmetrically situated on opposite sides of a central axis, wherein each of the first and second padded regions include a beveled inner surface adjacent the central axis, such that the first and second padded regions are separated by an inner notch formed by the beveled inner surfaces, and the first and padded regions are hingedly connected at the central axis;
a lower fastening region extending downward from a bottom end of the padded area along the central axis;
first and second straps extending outward, at a non-zero angle, from a top end of the padded area, wherein the first and second straps include distal strap ends; and
a lower fastening region configured to receive the distal strap ends, such that, when wrapped around the back of a user's knee, the inner notch of the padded area is aligned with, but does not contact, a scar on a user's knee.

2. The kneepad apparatus of claim 1, wherein each of the beveled inner edges have a bevel angle of approximately 40-50 degrees, such that the inner notch subtends a total angle of approximately 80-100 degrees.

3. The kneepad apparatus of claim 1, wherein the first and second straps extend from the top end of the padded area at an angle of approximately 10-20 degrees.

4. The kneepad apparatus of claim 1, wherein the distal strap ends connect to the lower fastening region via hook-and-loop fastening regions.

5. The kneepad apparatus of claim 1, wherein the first and second padded regions include foam core inserts having a thickness of approximately 0.8-1.2 inches.

6. The kneepad apparatus of claim 5, wherein each of the foam core inserts include horizontal troughs oriented perpendicular to the central axis.

7. The kneepad apparatus of claim 6, wherein the horizontal troughs include three troughs beveled at approximately a 45-degree angle and having a depth of approximately 0.25 inches.

8. The kneepad apparatus of claim 1, wherein the kneepad body comprises a Lycra fabric material.

9. The kneepad apparatus of claim 1, wherein the first and second straps comprise a Cordura fabric material.

10. A kneepad apparatus configured to be worn by a user having a vertical knee scar, the kneepad apparatus comprising:
a kneepad body having a first side configured to rest against a user's knee, and a second side opposite the first side;
a padded area provided on a first side of the kneepad body, the padded area including a first padded region and a second padded region symmetrically situated on opposite sides of a central axis;
wherein each of the first and second padded regions include a beveled inner surface adjacent the central axis, such that the first and second padded regions are separated by an inner notch having an angle of 80-100 degrees formed by the beveled inner surfaces, and the first and padded regions are hingedly connected at the central axis;
wherein the first and second padded regions include foam core inserts having a thickness of approximately 0.8-1.2 inches; and
wherein each of the foam core inserts include at least one horizontal trough oriented perpendicular to the central axis;
a lower fastening region extending downward from a bottom end of the padded area along the central axis;
first and second straps extending outward at an angle of approximately 10-20 degrees from a top end of the padded area, wherein the first and second straps include distal strap ends; and
a lower fastening region including a hook-and-loop fastener configured to receive the distal strap ends, such that, when wrapped around the back of a user's knee, the inner notch of the padded area is aligned with, but does not contact, the vertical scar on the user's knee.

11. The kneepad apparatus of claim 10, wherein the kneepad body comprises a Lycra fabric material.

12. The kneepad apparatus of claim 10, wherein the first and second straps comprise a Cordura fabric material.

13. A method of making a kneepad apparatus, the method comprising:
forming, from a single piece of stretch fabric, a first pattern piece;
folding the first pattern piece around a pair of foam inserts and sewing the first pattern piece to form a kneepad body having a first side configured to rest against a user's knee, and a second side opposite the first side;
wherein the first side of the kneepad body has a padded area including a first padded region and a second padded region symmetrically situated on opposite sides of a central axis, wherein each of the first and second padded regions include a beveled inner surface adjacent the central axis, such that the first and second padded regions are separated by an inner notch formed by the beveled inner surfaces, and the first and padded regions are hingedly connected at the central axis;
wherein the body further includes a lower fastening region extending downward from a bottom end of the padded area along the central axis;
attaching first and second straps extending outward, at a non-zero angle, from a top end of the padded area, wherein the first and second straps include distal strap ends and are configured to connect to a lower fastening region of the kneepad body such that, when wrapped around the back of a user's knee, the inner notch of the padded area is aligned with, but does not contact, a scar on a user's knee.

14. The method of claim 13, wherein each of the beveled inner edges have a bevel angle of approximately 40-50 degrees, such that the inner notch subtends a total angle of approximately 80-100 degrees.

15. The method of claim 13, wherein the first and second straps are formed to extend from the top end of the padded area at an angle of approximately 10-20 degrees.

16. The method of claim 13, wherein the distal strap ends are formed to connect to the lower fastening region via hook-and-loop fastening regions.

17. The method of claim 13, wherein the first and second padded regions include foam core inserts having a thickness of approximately 0.8-1.2 inches.

18. The method of claim 13, wherein each of the foam core inserts include horizontal troughs oriented perpendicular to the central axis.

19. The method of claim 18. wherein the horizontal troughs include three troughs beveled at approximately a 45-degree angle and having a depth of approximately 0.25 inches.

20. The method of claim 13, wherein the kneepad body comprises a Lycra fabric material, and the first and second straps comprise a Cordura fabric material.
